(19) United States
(12) Patent Application Publication
(10) Pub. No.: US 2007/0249000 A1
(43) Pub. Date: Oct. 25, 2007

(54) METHOD FOR DIAGNOSING A PERSON HAVING B-CELL PATHOLOGIES

(76) Inventors: Daniel Tuse, Vacaville, CA (US); Kevin Dawson, Sacramento, CA (US); William F. Haddon, Corte Madera, CA (US); Earl L. White, Fairfield, CA (US); Wasyl Malyj, Davis, CA (US)

Correspondence Address: LARGE SCALE BIOLOGY CORPORATION, 3333 VACA VALLEY PARKWAY, SUITE 1000, VACAVILLE, CA, 95688, US

(21) Appl. No.: 11/740,272

(22) Filed: Apr. 25, 2007

Related U.S. Application Data
(60) Provisional application No. 60/794,929, filed on Apr. 25, 2006.

(51) Int. Cl. G01N 33/574; G01N 33/50

(52) U.S. Cl. 435/7.23; 436/86

(57) ABSTRACT

Described is a method for diagnosing a person having or being at risk of developing certain B-cell pathologies, including Sjögren's Syndrome and non-Hodgkin's lymphoma, and excluding patients with symptoms similar to Sjögren's Syndrome but with a different etiology, comprising the following steps: providing a sample of a body fluid or tissue from said person, said sample containing a mixture of unknown proteins, protein fragments or peptides; analyzing said samples with mass spectrometry to generate a m/z (mass to charge ratio) spectrogram for each sample; comparing whether the patient's sample contains m/z values that are characteristic of a Sjögren's Syndrome reference database derived from the analysis and cataloguing of multiple patient spectrograms; and determining whether said patient either has or does not have Sjögren's Syndrome on the basis of this comparative analysis.

### FIELD OF THE INVENTION

The present invention relates to testing for risk factors of disease, and relates more particularly to risk factors for B-cell non-Hodgkin's lymphoma

### BACKGROUND OF THE INVENTION

The invention relates to a method for diagnosing a person having certain pathologies in which the behavior or function of B cells of the immune system, including B-cell neogenesis or regulation, may be responsible for certain diseases, such as Sjögren's Syndrome or B-cell non-Hodgkin's lymphoma. Further, the invention relates to a method for excluding a person as having Sjögren's Syndrome or certain lymphoproliferative diseases, so that appropriate diagnosis and treatment of a disease with similar symptoms but different causality or mechanism can be performed.

Sjögren's Syndrome is a common yet largely under-diagnosed autoimmune disease with B-cell involvement, afflicting millions of people. Because of the progressive nature of this disease, current diagnostics methods rely largely on monitoring a worsening of a patient's symptoms over a period of several years. Sjögren's Syndrome is characterized by a loss of function of the cells producing certain lubricating fluids in the body. Common symptoms of the disease may include dry eye and/or dry mouth, which account for the more obvious and more detectable manifestations, but other organs may be affected, such as internal organs or the nervous system, leading to moderate to severe impairment and pain in the affected individual.

Several clinical studies have demonstrated a relationship between Sjögren's Syndrome and lymphoproliferative disorders (see, for example, Fox, R I, 2005; Hansen et al., 2005; Szodoray and Jonsson, 2005; and Prochorec-Sobieszek and Wagner, 2005). Sjögren's Syndrome is a chronic autoimmune disorder of the exocrine glands with associated lymphocytic infiltrates of the affected glands. Dryness of the mouth and eyes results from involvement of the salivary and lachrymal glands. The exocrinopathy can be encountered alone, in which case the disease is termed primary Sjögren's Syndrome, or in the presence of another autoimmune disorder such as rheumatoid arthritis (RA), systemic lupus erythematosus (SLE), or progressive systemic sclerosis (PSS) (Fox, R I, Lancet 2005. Jul 23-29; 366 (9482): 321-331). Current diagnosis is multifactorial, but usually requires objective signs and symptoms of dryness including a characteristic appearance of a biopsy sample from a minor salivary gland or autoantibody such as anti-Sjögren's Syndrome antibody (“anti-SS-A”). Exclusions to the diagnosis include infections with HIV, human T-lymphotropic virus type I, or hepatitis C.

B-Cell proliferation is a characteristic of Sjögren's Syndrome, with lesions that range from benign to malignant. In fact, it has been found in epidemiological studies that patients that present with symptoms of Sjögren's Syndrome have a forty fold (40-fold) increased risk of developing B-cell non-Hodgkin's lymphoma (Prochorec-Sobieszek, M., and Wagner, T., Otolaryngol. Pol., 2005; 59 (4): 559-564). When lymphomas complicate the course of Sjögren's Syndrome, they arise in mucosal extranodal sites, especially in the salivary gland, and are usually slowly progressive (indolent) lymphomas. The main difficulty in salivary lymphoproliferation in Sjögren's Syndrome is borne from the differential diagnosis of lymphoma; thus, the simple detection of B-cell clonality cannot be used as a criterion for the diagnosis of B-cell malignancies in a background complicated by Sjögren's Syndrome.

Thus, there is a need to differentiate patients suffering primary Sjögren's Syndrome from those with Sjögren's Syndrome plus accompanying autoimmune disorders such as RA, SLE, or PSS, and those with Sjögren's Syndrome accompanied by more serious conditions such as lymphomas, which themselves may be difficult to diagnose, especially at the early stages (Grulich, A E and Vajdic, C M, Pathology, 2005 December; 37 (6):409-419). The value of more specific, selective, and accurate diagnoses in these diseases sharing some commonality of symptoms is to offer the patient the most relevant course of therapy to safely and effectively manage the course of each disease.

### SUMMARY

In a first embodiment we present a method for diagnosing a person suspected of having B-cell non-Hodgkin's lymphoma or at risk of developing B-cell non-Hodgkin's lymphoma. The steps of the method are providing a sample of a body fluid or a tissue from said person, said sample containing a mixture of uncharacterized proteins, peptides or protein fragments naturally occurring in the sample; pre-processing the patient sample as necessary to make it amenable to analysis by mass spectrometry; generating a mass spectrum of the protein-, protein fragment- and peptide-containing patient sample; applying mathematical algorithm(s) to differentiate whether the mass spectrum of the sample has features, biomarkers, or patterns of biomarkers that are characteristic of, or in common with, other samples, similarly processed and analyzed, and derived from persons known to have Sjögren's Syndrome (Sjögren's Syndrome “fingerprint” or reference database); and diagnosing the high risk in the person for developing B-cell non-Hodgkin's lymphoma depending on whether the person's sample is classified by the algorithm(s) into the Sjögren's Syndrome fingerprint or reference database relative to non-Sjögren's Syndrome-derived samples.

A sample used in the first embodiment can be obtained from human blood, plasma, serum, saliva, tears, lymph, urine, cerebrospinal fluid, any biopsy material or tissue sample, including bone marrow, lymph nodes, nervous tissue, skin, hair, fetal material including amniocentesis material, uterine tissue, feces or semen.

In the first embodiment, classification of a sample into one belonging to a patient at risk or having a propensity of developing B-cell non-Hodgkin's lymphoma can be determined by the existence of or the predominance of features, biomarkers, or patterns of biomarkers in common with a Sjögren's Syndrome fingerprint or reference database, said biomarkers being defined as mass-to-charge ratios (m/z) generated by mass spectrometry for one or more proteins, protein fragments or peptides present in the sample.

In the first embodiment, classification of a sample into one belonging to a patient at risk or having a propensity of developing B-cell non-Hodgkin's lymphoma can be determined by the absence of or the relative lack of features, biomarkers, or patterns of biomarkers relative to non- Sjögren's Syndrome-derived samples, said biomarkers being defined as mass-to-charge ratios (m/z) generated by mass spectrometry for one or more proteins, protein fragments or peptides present in the sample.

In the first embodiment, biomarkers characteristic of the Sjögren's Syndrome fingerprint or reference database include, but are not limited to, those having an m/z values of 902.48, 1,479.76, 2,407.30, 2,536.16, 3,655.78, 4,281.14, 4,930.28, 5,843.94, and 5,942.00.

A biomarker characteristic of the Sjögren's Syndrome fingerprint or reference database is preferably one of the doubly charged ions in the m/z range of 950 to 4,000, and most preferably in the m/z range of 3,500 to 3,950.

A biomarker characteristic of the Sjögren's Syndrome fingerprint or reference database is preferably one of the doubly charged ions in the m/z m/z range of 3,803 to 3,808.

A doubly charged ion in the m/z range of 3,500 to 3,950, and preferably in the m/z range of 3,803 to 3,808, can be diagnostic for Sjögren's Syndrome.

In the first embodiment, further assurance can be obtained by determining all or a portion of a nucleic acid sequence encoding the protein, protein fragment or peptide associated with Sjögren's Syndrome and thereby with a person's having, or propensity or predisposition to developing, B-cell non-Hodgkin's lymphoma, and determining the presence or level of said sequence through DNA or RNA analysis.

In the first embodiment, nucleic acids encoding any Sjögren's Syndrome-associated proteins, protein fragments or peptides that are indicative of the existence of or the propensity or predisposition of a person to developing B-cell non-Hodgkin's lymphoma, are detected by a method such as single-strand conformation polymorphism (SSCP) analysis, restriction analysis, microarray technology, nucleic acid amplification method such as polymerase chain reaction method.

The presence of Sjögren's Syndrome-associated, and thus B-cell non-Hodgkin's lymphoma existing or predisposing, biomarkers, fingerprints, proteins, protein fragments, peptides or nucleic acids, can be performed within a screening test.

A simplified method can be put into kit form. The kit could contain means for detecting the presence of Sjögren's Syndrome-associated proteins, protein fragments, peptides or nucleic acids, and thereby assessing a person's having or propensity or predisposition to developing B-cell non-Hodgkin's lymphoma. A patient sample would be mixed with the contents of a vial in the kit.

Such a Kit assay can use antibodies or peptides including mutation-specific antibodies, ELISA, Western Blotting assays, flow cytometry assays and assays using immunohistochemical techniques including confocal microscopy, or nucleic acid amplification methods, single-strand conformation polymorphism (SSCP) analysis, restriction analysis, microarray technology and other available assays.

In another embodiment for diagnosing a person suspected of having B-cell non-Hodgkin's lymphoma or at risk of developing B-cell non-Hodgkin's lymphoma, the steps include providing a sample of a body fluid or a tissue from said person, said sample containing a mixture of uncharacterized proteins, peptides or protein fragments naturally occurring in the sample; differentiating whether the sample has features biomarkers, or patterns of biomarkers that are characteristic of, or in common with, other samples, similarly processed and analyzed, and derived from persons known to have Sjögren's Syndrome (Sjögren's Syndrome “fingerprint” or reference database) by analyzing a protein, protein fragment or peptide associated with Sjögren's Syndrome or its nucleic acid by employing any or a combination of methods selected from the group consisting of chromatography, mass spectrometry, radioimmunoassay, ELISA, plasmon resonance spectroscopy, protein sequencing, biosensors, protein chips, nucleic acid amplification method, single-strand conformation polymorphism (SSCP) analysis, restriction analysis, microarray technology and a mathematical algorithm; and diagnosing from the information derived in step b whether the person is a risk or at an increased risk for developing B-cell non-Hodgkin 's lymphoma depending on whether the person's sample is classified into the Sjögren's Syndrome fingerprint or reference database relative to non-Sjögren's Syndrome-derived samples.

A sample used in this embodiment can also be obtained from human blood, plasma, serum, saliva, tears, lymph, urine, cerebrospinal fluid, any biopsy material or tissue sample, including bone marrow, lymph nodes, nervous tissue, skin, hair, fetal material including amniocentesis material, uterine tissue, feces or semen.

Classification of a sample into one belonging to a patient at high risk or propensity of developing B-cell non-Hodgkin's lymphoma can be determined by the existence of or the predominance of features, biomarkers, or patterns of biomarkers in common with a Sjögren's Syndrome fingerprint or reference database, said biomarkers being defined as mass-to-charge ratios (m/z) generated by mass spectrometry for one or more proteins, protein fragments or peptides present in the sample. Classification of a sample into one belonging to a patient at high risk or propensity of developing B-cell non-Hodgkin's lymphoma can also be determined by the absence of or the relative lack of features, biomarkers, or patterns of biomarkers relative to non- Sjögren's Syndrome-derived samples, said biomarkers being defined as mass-to-charge ratios (m/z) generated by mass spectrometry for one or more proteins, protein fragments or peptides present in the sample.

Differentiation between samples can be performed by analyzing for the presence or absence of said biomarker characteristic of the Sjögren's Syndrome fingerprint or its encoding nucleic acid wherein the biomarkers include, one or more proteins, protein fragments, or peptides of the group consisting of m/z values of 902.48, 1,479.76, 2,407.30, 2,536.16, 3,655.78, 4,281.14, 4,930.28, 5,843.94, and 5,942.00 and at least one antibody to one or more proteins, protein fragments, or peptides of the group consisting of m/z values of 902.48, 1,479.76, 2,407.30, 2,536.16, 3,655.78, 4,281.14, 4,930.28, 5,843.94, and 5,942.00

Preferably a doubly charged biomarker characteristic of the Sjögren's Syndrome fingerprint is selected from a group in the m/z range of 3,803 to 3,808 and at least one antibody, single-chain-antibody or other binding agent having specificity to one or both of the said biomarkers will be used.

### DETAILED DESCRIPTION OF THE INVENTION

Attempts to diagnose primary Sjögren's Syndrome or Sjögren's Syndrome complicated by other preexisting autoimmune or lymphoproliferative conditions using simple diagnostic procedures have not been successful. A primary reason for this difficulty in accurate diagnosis has been the lack of correlating biological indicators of the disease (“markers” or “biomarkers”) amidst a population of patients that presents with widely varying degrees of pathology, in a progressive conditions that may worsens over time in a patient-specific manner.

Because perhaps the most obvious symptoms in people putatively suffering from Sjögren's Syndrome is the lack of tear and saliva production, studies have tried to differentiate the content of tears or saliva of patients with Sjögren's Syndrome from those obtained from normal (healthy) donors.

Tomosugi et al. (Tomosugi et al., J. Proteome Res. 2005 May-June; 4(3): 820-825) reported the diagnostic potential of tear proteomic patterns in Sjögren's Syndrome, based on their assumption that histological and functional changes of the lachrymal gland might be reflected in proteomic (protein profile) patterns in tear fluids. In this study, the protein profiles of tears from thirty one (31) patients with primary Sjögren's Syndrome and fifty seven (57) control (healthy) samples were analyzed by SELDI-TOF-MS (surface-enhanced laser desorption/ionization time-of-flight mass spectrometry) and compared. Multiple protein changes were reproducibly detected in the primary Sjögren's Syndrome group, including 10 potentially novel biomarkers. Seven of the biomarkers (m/z values of: 2094, 2743, 14191, 14702, 16429, 17453, 17792) were down-regulated (expressed in lower concentration) in the Sjögren's Syndrome group relative to the control group, and three biomarkers (m/z values of: 3483, 4972, 10860) were up-regulated (expressed in higher concentration) in the disease group relative to the control group. These investigators reported a sensitivity score of 87% and a specificity score of 100% in their analyses. For reference, the higher the sensitivity value the lower the chance that a diagnostic procedure could yield a false-negative result, and the higher the specificity value the lower the chance a diagnostic procedure could yield a false-positive result.

Similar studies have compared tear protein expression using two-dimensional electrophoresis (2-D PAGE), a more traditional technique for proteomic analysis. For example, Koo et al. (Koo et al., J. Proteome Res. 2005 May-June; 4(3): 719-724) studied the tear proteome in samples from nineteen (19) normal (healthy) volunteers and samples obtained from twenty seven (27) patients with chronic blepharitis, one of the most common conditions seen in the opthalmologist's office. These investigators reported differences in the protein patterns from each group, and attempted to identify the differentially expressed proteins by the technique of ESI-Q-TOF (electrospray-quadrupole time-of-flight mass spectrometry) and confirmed their findings with Western blotting. They reported that mine (9) proteins in the patient samples were down-regulated about 50% relative to the healthy donor group, and identified 8 of the 9 proteins. This study shows that once differentially expressed biomarkers are found through proteomic analysis, techniques exist to identify the individual proteins, protein fragments or peptides (“molecular markers”) corresponding to each biomarker. Although not stated in this study, it is generally known to those skilled in the art that once a protein, protein fragment or peptide is identified, either through techniques such as those described by Koo et al. or by protein sequencing, the gene sequences encoding their synthesis can also be identified by searching available public or private genomic databases.

While analysis of tear fluid appears to be a promising technique in the study of Sjögren's Syndrome, the collection of tear fluid from patients that have difficulty generating tears due to their disease could lead to poor sample collection, or inflammation or other complications for the patient. Also, the small sample size could introduce variability into the analysis.

To overcome this challenge, other investigators have focused on the proteomic analysis of saliva samples, which can be more readily obtained and in larger quantities, with minimal subject discomfort. For example, Ryu and co-workers (Ryu et al., 2006, Mar. 7; Rheumatology (Oxford); Epub ahead of print), analyzed the protein profiles of parotid salivary gland fluid from Sjögren's Syndrome patients and healthy donors (controls) by also using the technique of SELDI-TOF-MS, coupled with 2-D DIGE (two-dimensional difference gel electrophoresis). In this study, samples of saliva from the parotid gland of forty one (41) Sjögren's Syndrome patients and twenty (20) healthy control subjects were analyzed and compared. As reported in other studies, some proteins were increased in patients relative to healthy volunteers while others were decreased. Greater than two-fold (>2-fold) increases in peaks representing MW (molecular weights) of 11.8, 12.0, 14.3, 80.6 and 83,7 kDa were reported, while decreases in MW of 17.3, 25.4 and 35.4 kDa in Sjögren's Syndrome samples were noted. Among the candidate biomarker proteins up-regulated in the Sjögren's Syndrome group were: beta-2-microglobulin, lactoferrin, immunoglobulin (Ig) kappa light chain, polymeric Ig receptor, lysozyme C and cystatin C, while down-regulated candidate proteins included: amylase and carbonic anhydrase VI. While these investigators published their observations, they did not report the sensitivity, specificity or accuracy of their molecular marker-based model, and could not justify their methodology as diagnostic for Sjögren's Syndrome.

Regardless of whether proteins samples for analysis are obtained from tears, saliva, serum or other bodily fluids or tissue biopsies, the analytical method used is often a determining factor in whether reproducible differences in the proteome of sample groupings can be obtained with sufficient sensitivity, specificity and accuracy to be of diagnostic value. Even if samples are rigorously collected, processed and stored, techniques such as SELDI are known to those skilled in the art to be of relatively low resolution. Early work in this field published by Petricoin, Liotta and collaborators (Petricoin et al., 2002) attempted to demonstrate that disease-associated biomarkers could be detected through the use of SELDI MS. However, the SELDI technique used by Petricoin et al. had a relatively poor resolution of 1 part in 200 Daltons (mass-to-charge units), putting greater emphasis on the intensity of the SELDI signal to differentiate among candidate peaks. The importance of this limitation is evident from studies such as Kozak et al. (Kozak et al. 2003, FIG. 1), wherein the required accuracy of measurement of intensity values appears to have exceeded the reproducibility of the methods used to extract peptides and proteins from the serum samples. Diamandis (Diamandis EP, 2004) provides a more comprehensive review of these types of problems with SELDI and other forms of MS applied to diagnostics and biomarker discovery. With such low resolutions, SELDI is bound to miss (ie, not detect) some important biomarkers, the up- or down-regulation of which may be key to the diagnosis of Sjögren's Syndrome, or its differentiation from other diseases with similar symptomatology but whose unrelated etiology could require different clinical management options. In contrast, MALDI (matrix-assisted laser desorption/ionization) MS, which was used in the present invention, can be tuned to very high resolution in some instruments, and relative mass resolutions of 15,000 (compared to SELDI's mass resolution of 200) are not uncommon (PerkinElmer Protof 2000 instrument specifications and Predictive Diagnostics, Inc.'s, direct empirical experience). This mass resolution allows for discovery of biomarkers with much higher signal-to-noise ratios.

ESI (electrospray ionization) MS, an alternate MS-based method for biomarker detection, is also known to those skilled in the art to be prone to introducing redundancy into the mass spectrum, thus lowering the information content of the spectrum, because proteins present in the sample display at many masses due to multiple charging. For example, data presented by Correlogic Systems Inc. (Bethesda, Md.) at the American Society of Clinical Oncologists (ASCO) Annual Meeting, Orlando Fla., May 15, 2005, “A Serum Pattern Predictive of Breast Cancer,” clearly show that the peculiarities of the ESI method cause a crowding of the lower end of the mass spectrum by multiply charged ions, obscuring putative biomarkers which could otherwise have been visualized. Other techniques, such as those relying on CZE (capillary zone electrophoresis) MS, may require complex sample pre-processing for generating accurate spectra. With CZE as well as with other MS techniques, the introduction of proteases or other reagents to the sample in order to cleave patient-derived proteins into fragments to assist with mass spectral analysis can introduce noise and complexity into spectra, further complicating analysis and data interpretation (see, for example, Villanueva et al., 2006).

Thus, there remains an unmet need for simple and accurate methods for diagnosing Sjögren's Syndrome based on minimally invasive sample collection procedures, minimum sample processing without introduction of artifacts or additional complexity of content, and for differentiating this disease reliably from others with similar symptomatology but different etiology and treatment options.

Methods to diagnose a disease or condition based on individual, identifiable molecular markers (ie. proteins, other analytes) in a sample are typical and traditional in the diagnostics industry and well known to those skilled in the art. Such methods rely on comparisons of the presence, absence, or relative concentration or level of one or more analytes in a subject's sample relative to the level or concentration of the analyte in a pooled weighted average for normal or healthy subjects. Such molecular markers usually are positively linked to a disease or condition. This type of traditional diagnostic strategy can be called “hypothesis-driven”, in the sense that a lot must be known about the disease itself, and the genes or proteins that form the basis of the analyte and which correspond directly with the disease or condition must be isolated, defined, well characterized and correlated to the disease in exhaustive clinical validation trials.

An alternative diagnostic strategy can be called “discovery based”, because it relies on discovery of biomarkers with diagnostic value which are found purely from empirical analysis of protein or other samples from patients suffering a disease or condition and from healthy (control) donors. In this strategy, there is no bias regarding whether a protein or other marker must be derived directly from a diseased tissue or process. Furthermore, very large, multivariate sample sets can be accommodated in this strategy because the power of this method is in its ability to analyze multiple markers, whether nor not they are directly related to a diseased tissue or process. This approach has been successfully applied, for example, as described in Anderson et al. (US patent U.S. Pat. No. 6,980,674).

The current diagnosis of Sjögren's Syndrome involves a complex series of procedures, and interpretation of results and subsequent categorization of a patient as having Sjögren's Syndrome, or an unrelated disease with similar symptomatology, is subjective, protracted and not accurate. Thus, Sjögren's Syndrome is one of several types of diseases that are very difficult to characterize or diagnose through conventional methods that rely on molecular markers, and where discovery based, multivariate biomarker analysis may offer a new diagnostic solution.

To date no clear marker has been reported for Sjögren's Syndrome, and mere recognition of Sjögren's Syndrome, or exclusion of a patient as having Sjögren's Syndrome so that appropriate interventions for his or her actual affliction can be pursued, would be very beneficial for early onset of therapy or preventive measures.

It is therefore an object of the present invention to provide an efficient diagnosis system for Sjögren's Syndrome, giving a clear indication and a clear correlation to this disease or to the lack of this disease.

The subject matter of the invention is therefore a method for diagnosing a person having Sjögren's Syndrome or for excluding a person as having Sjögren's Syndrome, characterized by the following steps:

providing a sample of a body fluid or a tissue from said person, said sample containing a mixture of uncharacterized proteins, peptides or protein fragments naturally occurring in the sample,

pre-processing the patient sample as necessary to make it amenable to analysis by mass spectrometry,

generating a mass spectrum of the protein-, protein fragment- and peptide-containing patient sample,

applying mathematical algorithms to differentiate whether the mass spectrum of the sample has features, markers, or patterns of markers that are characteristic of, or in common with, other samples, similarly processed and analyzed, and derived from persons known to have Sjögren's Syndrome (Sjögren's Syndrome “fingerprint”),

Diagnosing Sjögren's Syndrome in the person or excluding the person as having Sjögren's Syndrome, depending on whether the person's sample fits the Sjögren's Syndrome “fingerprint” criteria.

In the present invention, the technique of MALDI TOF MS (matrix-assisted, laser desorption/ionization time-of-flight mass spectrometry) was used to characterize the proteins contained in samples of saliva obtained from patients positively diagnosed as having Sjögren's Syndrome and from subjects who did not have the disease (healthy/control group).

Mass spectra were analyzed by a series of mathematical algorithms, some of which evaluated and rejected outlier spectra and others which normalized baseline features or enhanced the signal to noise ratios. The resulting processed spectra were analyzed via other algorithms which identified consensus features in the patient and control group samples, from which a reference database (ie. a “Sjögren's Syndrome fingerprint”) was built to differentiate Sjögren's Syndrome samples from control samples.

This method enabled us to successfully build several mathematical models to characterize Sjögren's Syndrome biomarkers. One such classification model (Model 1) produced seven putative biomarkers, with a sensitivity of 97.5%, a specificity of 97.8% and an accuracy of 97.6%. Biomarkers identified were detected at m/z values of: 902.48, 2407.30, 2912.56, 3655.78, 3803.38, 4281.14 and 5942.00. Another set of seven putative biomarkers was identified using a separate classification model (Model 2). This model yielded sensitivity, specificity and accuracy values of 85.0%, 91.3% and 88.1%, respectively. Biomarkers identified with this model were at m/z values of: 902.48, 1479.76, 2536.16, 3655.78, 3803.38, 4930.28 and 5843.94. The three (3) biomarkers underlined were commonly found using both models.

Surprisingly, our mathematical algorithms revealed the presence of a doubly charged ion present only in the Sjögren's Syndrome-derived patient samples. This biomarker had mono-isotopic peaks every ½ atomic mass unit in the mass-to-charge (m/z) range of 3,803 to 3,808. Because this biomarker was exclusively present in the Sjögren's Syndrome group, it could be useful as a diagnostic endpoint for this difficult-to-diagnose disease. In addition to the biomarker's utility as a diagnostic tool, the protein, protein fragment or peptide and its/their encoding nucleic acid sequence(s) could also be useful as molecular or genetic markers, respectively, in the diagnosis of patients with Sjögren's Syndrome or in the exclusion of patients with similar symptomatology.

### EXAMPLES

### Example 1

A total of twenty seven 27 (14 primary and 13 secondary) Sjögren's Syndrome patients and 27 age-matched healthy controls (non-Sjögren's Syndrome subjects) were recruited for these studies by a collaborative team from The Schepens Eye Research Institute (SERI) and the Tufts University School of Medicine (“SERI/Tufts”). Non-stimulated submandibular glands saliva was collected from the Wharton's duct using a suction device. Two □l of salvia were diluted in 180 □l of 0.2% trifluoroacetic acid and processed for mass spectrometry analyses. Mass spectra were acquired on a prOTOF 2000 matrix-assisted laser desorption/ionization orthogonal time of flight (MALDI-O-TOF) mass spectrometer in the molecular weight range of 750-12,000 Da. Raw data were exported and sent for analysis by Predictive Diagnostics Inc. (PDI, Vacaville, Calif.) who utilized proprietary bioinformatics tools to identify biomarkers.

### Example 2

Spectra generated by SERI/Tufts were transmitted to PDI via Secure Socket Layer upload. Triplicate spectra were generated for each sample, for a total of 153 spectra. For the most part, the mass spectra appeared to be of high-quality but low intensity throughout the m/z range of 751-11,951. All spectra were evaluated as entire spectra and also in 6 steps, each focusing on one of six m/z ranges. The algorithms we used identified 4 outlier spectra, 3 of which originating from the same subject. This subject and the fourth outlier spectrum were removed before further analysis. The remaining 149 spectra were background-corrected and average spectra were computed for each of the 50 remaining subjects.

### Example 3

Pattern recognition and feature selection algorithms were applied to detect spectral features distinguishing the two groups of subjects. Our algorithms successfully produced several classification models. Model performance was evaluated using 50-fold cross-validation and the model sensitivity/specificity parameters were reported. We report two models with high sensitivity and specificity. Both models are composed of 7 markers, 3 of which are present in both models.

More specifically, mathematical analysis on the two SERI/Tufts sample sets enabled PDI to build several classification models, which in turn resulted in the identification of several biomarkers. A model based on seven putative biomarkers (Model 1) yielded a sensitivity of 97.5%, specificity of 97.8% and an accuracy of 97.6%. Biomarkers identified were detected at m/z values 902.48, 2,407.30, 2,912.56, 3,655.78, 3,803.38, 4,281.14, and 5,942.00. Model performance parameters were evaluated using 50-fold cross-validation. Identified biomarkers may or may not represent the mono-isotopic masses of the protein/peptide fragments.

Another set of seven putative biomarkers was identified in another classification model (Model 2). This model yielded a sensitivity of 85.0%, specificity of 91.3%, and an accuracy of 88.1%. Biomarkers identified in this model were detected at m/z values 902.48, 1,479.76, 2,536.16, 3,655.78, 3,803.38, 4,930.28, and 5,843.94. Three of the biomarkers (underlined) were common to both models.

In the accompanying figures, we individually show the m/z regions around all of these markers. Each biomarker is presented in four panels. One panel shows the individual spectra, the second panel shows the heat-map of the 50 spectra, a third panel shows a visually enhanced heat-map emphasizing the m/z regions different between the two groups, and finally, the fourth panel summarizes the two populations of spectra viewing the median and the 25-75 percentile range for both groups.

Unexpectedly, our algorithms revealed one biomarker that is an obvious doubly-charged ion present only in group 1, the Sjögren's Syndrome group. This biomarker has mono-isotopic peaks every ½ atomic mass unit in the m/z range 3803-3808. As expected, the singly-charged ion can also be seen in the m/z range 7606-7616.

Because Sjögren's Syndrome patients are at higher risk of developing B-cell lymphoma, this same procedure can be used to determine whether a person is at an increased risk of developing a B-cell lymphoma. Accordingly we offer this method to determine whether a patient is at an increased risk of developing B-cell lymphoma.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### REFERENCES

Ryu O H, Atkinson J C, Hoehn G T, Illei G G, Hart T C. Identification of parotid salivary biomarkers in Sjögren's syndrome by surface-enhanced laser desorption/ionization time-of-flight mass spectrometry and two-dimensional difference gel electrophoresis. Rheumatology (Oxford). 2006 Mar. 7; [Epub ahead of print]

Petricoin E M, A. Ardekani M, Hitt B A, Levine P J, Fusaro V A, Steinberg S M, Mills G B, Simone C, Fishman D A, Kohn E C, and Liotta L A. Use of Proteomic Patterns In Serum to Identify Ovarian Cancer. 2002. The Lancet, 359, 572-575.

Kozak, K R, Amneus M W, Pusey S M, Su F, Luong M N, Luong S A, Reddy S T and Farias-Eisner R. Identification of Biomarkers for Ovarian Cancer Using Strong Anion-Exchange Protein Chips: Potential Use in Diagnosis and Prognosis. Proc. Nat. Acad. Sci., 2003, 100, 12343-48 (esp. FIG. 1).

Diamandis E P. Mass Spectrometry as a Diagnostic and a Cancer Biomarker Discovery Tool: Opportunities and Potential Limitations. Mol Cell Proteomics 2004, 3(4), 367-78.

Correlogic Systems Inc. (Bethesda, Md.), presentation at the American Society of Clinical Oncologists (ASCO) Annual Meeting. “A Serum Pattern Predictive of Breast Cancer.” ASCO Annual Meeting, Orlando, Fla., May 15, 2005.

Villanueva J, Shaffer D R, Philip J, Chaparro C A, Erdjument-Bromage H, Olshen A B, Fleisher M, Lilja H, Boyd J, Sanchez-Carbayo M, Holland E C, Cordon-Cardo C, Scher H, and Tempst P. Differential Exoprotease Activities Confer Tumor-Specific Serum Peptidome Patterns. J. Clinical Investigation, 2006, 116(1), 271-84.

Tomosugi N, Kitagawa K, Takahashi N, Sugai S, Ishikawa I. Diagnostic potential of tear proteomic patterns in Sjögren's syndrome. J. Proteome Res. 2005 May-June;4(3):820-5.

Koo B S, Lee D Y, Ha H S, Kim J C, Kim C W. Comparative analysis of the tear protein expression in blepharitis patients using two-dimensional electrophoresis. J. Proteome Res. 2005 May-June;4(3):719-24.

Prochorec-Sobieszek M, Wagner T. Lymphoproliferative disorders in Sjögren's syndrome. Otolanyngol Pol. 2005;59(4):559-64.

Grulich A E, Vajdic C M. The epidemiology of non-Hodgkin lymphoma. Pathology. 2005 December; 37(6):409-19.

Szodoray P, Jonsson R. The BAFF/APRIL system in systemic autoimmune diseases with a special emphasis on Sjögren's syndrome. Scand J. Immunol. 2005 November; 62(5):421-8.

Hansen A, Lipsky P E, Dorner T. Immunopathogenesis of primary Sjogren's syndrome: implications for disease management and therapy. Curr Opin Rheumatol. 2005 September; 17(5):558-65.

Fox R I. Sjogren's syndrome. Lancet. 2005 Jul 23-29;366(9482):321-31.

What is claimed is:

1. A method for diagnosing a person suspected of having B-cell non-Hodgkin's lymphoma or at risk of developing B-cell non-Hodgkin's lymphoma, comprising:
a) providing a sample of a body fluid or a tissue from said person, said sample containing a mixture of uncharacterized proteins, peptides or protein fragments naturally occurring in the sample;
b) pre-processing the patient sample as necessary to make it amenable to analysis by mass spectrometry;
c) generating a mass spectrum of the protein-, protein fragment- and peptide-containing patient sample;
d) applying mathematical algorithm(s) to differentiate whether the mass spectrum of the sample has features, biomarkers, or patterns of biomarkers that are characteristic of, or in common with, other samples, similarly processed and analyzed, and derived from persons known to have Sjögren's Syndrome (Sjögren's Syndrome “fingerprint” or reference database); and
e) diagnosing the high risk in the person for developing B-cell non-Hodgkin 's lymphoma depending on whether the person's sample is classified by the algorithm(s) into the Sjögren's Syndrome fingerprint or reference database relative to non-Sjögren's Syndrome-derived samples.

2. A method according to claim 1 wherein said sample is derived from human blood, plasma, serum, saliva, tears, lymph, urine, cerebrospinal fluid, any biopsy material or tissue sample, including bone marrow, lymph nodes, nervous tissue, skin, hair, fetal material including amniocentesis material, uterine tissue, feces or semen.

3. A method according to claim 1 wherein said classification of a sample into one belonging to a patient at high risk or propensity of developing B-cell non-Hodgkin's lymphoma is determined by the existence of or the predominance of features, biomarkers, or patterns of biomarkers in common with a Sjögren's Syndrome fingerprint or reference database, said biomarkers being defined as mass-to-charge ratios (m/z) generated by mass spectrometry for one or more proteins, protein fragments or peptides present in the sample.

4. A method according to claim 1 wherein said classification of a sample into one belonging to a patient at high risk or propensity of developing B-cell non-Hodgkin's lymphoma is determined by the absence of or the relative lack of features, biomarkers, or patterns of biomarkers relative to non- Sjögren's Syndrome-derived samples, said biomarkers being defined as mass-to-charge ratios (m/z) generated by mass spectrometry for one or more proteins, protein fragments or peptides present in the sample.

5. A method according to claim 1, wherein the biomarkers characteristic of the Sjögren's Syndrome fingerprint or reference database include, but are not limited to, a group consisting of m/z values of 902.48, 1,479.76, 2,407.30, 2,536.16, 3,655.78, 4,281.14, 4,930.28, 5,843.94, and 5,942.00.

6. A method according to claim 4 wherein the biomarker characteristic of the Sjögren's Syndrome fingerprint or reference database is selected from a group of doubly charged ions in the m/z range of 950 to 4,000, and in the m/z range of 3,500 to 3,950.

7. A method according to claim 6 wherein a doubly charged biomarker characteristic of the Sjögren's Syndrome fingerprint or reference database is selected from a group in the m/z range of 3,803 to 3,808.

8. A method of claim 1, wherein the presence of a doubly charged ion in the m/z range of 3,500 to 3,950, and in the m/z range of 3,803 to 3,808, is diagnostic for Sjögren's Syndrome.

9. A method of claim 1, further comprising determining all or a portion of a nucleic acid sequence encoding the protein, protein fragment or peptide associated with Sjögren's Syndrome and thereby with a person's having, or propensity or predisposition to developing, B-cell non-Hodgkin's lymphoma, and determining the presence or level of said sequence through DNA or RNA analysis.

10. A method according to claim 9 wherein nucleic acids encoding any Sjögren's Syndrome-associated proteins, protein fragments or peptides, and therefore indicating the existence of or the propensity or predisposition of a person to developing B-cell non-Hodgkin's lymphoma, are detected by a method selected from the group consisting of a nucleic acid amplification method, single-strand conformation polymorphism (SSCP) analysis, restriction analysis, microarray technology.

11. A method according to claim 10, wherein said nucleic acid amplification method is a polymerase chain reaction method.

12. A method according to claim 1, wherein said detecting the presence of Sjögren's Syndrome-associated, and thus B-cell non-Hodgkin's lymphoma existing or predisposing, biomarkers, fingerprints, proteins, protein fragments, peptides or nucleic acids, is performed within a screening test.

13. Kit for performing any of the methods according to claims 1, comprising means for detecting the presence of Sjögren's Syndrome-associated proteins, protein fragments, peptides or nucleic acids, and thereby assessing a person's having or propensity or predisposition to developing B-cell non-Hodgkin's lymphoma.

14. Kit according to claim 13, wherein said assays to detect Sjögren's Syndrome-associated proteins, protein fragments, peptides or nucleic acids, and thereby assessing a person's having a high risk to developing B-cell non-Hodgkin's lymphoma, are selected from a group of assays consisting of using antibodies or peptides including mutation-specific antibodies, ELISA, Western Blotting assays, flow cytometry assays and assays using immunohistochemical techniques including confocal microscopy, or nucleic acid amplification methods, single-strand conformation polymorphism (SSCP) analysis, restriction analysis, microarray technology.

15. A method for diagnosing a person suspected of having B-cell non-Hodgkin's lymphoma or at risk of developing B-cell non-Hodgkin's lymphoma, comprising:
a) providing a sample of a body fluid or a tissue from said person, said sample containing a mixture of uncharacterized proteins, peptides or protein fragments naturally occurring in the sample;
b) differentiating whether the sample has features biomarkers, or patterns of biomarkers that are characteristic of, or in common with, other samples, similarly processed and analyzed, and derived from persons known to have Sjögren's Syndrome (Sjögren's Syndrome “fingerprint” or reference database) by analyzing a protein, protein fragment or peptide associated with Sjögren's Syndrome or its nucleic acid by employing any or a combination of methods selected from the group consisting of chromatography, mass spectrometry, radioimmunoassay, ELISA, plasmon resonance spectroscopy, protein sequencing, biosensors, protein chips, nucleic acid amplification method, single-strand conformation polymorphism (SSCP) analysis, restriction analysis, microarray technology and a mathematical algorithm; and
c) diagnosing from the information derived in step b whether the person is a risk or at an increased risk for developing B-cell non-Hodgkin's lymphoma depending on whether the person's sample is classified into the Sjögren's Syndrome fingerprint or reference database relative to non-Sjögren's Syndrome-derived samples.

16. A method according to claim 1 wherein said sample is derived from human blood, plasma, serum, saliva, tears, lymph, urine, cerebrospinal fluid, any biopsy material or tissue sample, including bone marrow, lymph nodes, nervous tissue, skin, hair, fetal material including amniocentesis material, uterine tissue, feces or semen.

17. A method according to claim 1 wherein said classification of a sample into one belonging to a patient at high risk or propensity of developing B-cell non-Hodgkin's lymphoma is determined by the existence of or the predominance of features, biomarkers, or patterns of biomarkers in common with a Sjögren's Syndrome fingerprint or reference database, said biomarkers being defined as mass-to-charge ratios (m/z) generated by mass spectrometry for one or more proteins, protein fragments or peptides present in the sample.

18. A method according to claim 1 wherein said classification of a sample into one belonging to a patient at high risk or propensity of developing B-cell non-Hodgkin's lymphoma is determined by the absence of or the relative lack of features, biomarkers, or patterns of biomarkers relative to non- Sjögren's Syndrome-derived samples, said biomarkers being defined as mass-to-charge ratios (m/z) generated by mass spectrometry for one or more proteins, protein fragments or peptides present in the sample.

19. A method according to claim 1, wherein differentiating is performed by analyzing for the presence or absence of said biomarker characteristic of the Sjögren's Syndrome fingerprint or its encoding nucleic acid wherein the biomarkers include, one or more proteins, protein fragments, or peptides of the group consisting of m/z values of 902.48, 1,479.76, 2,407.30, 2,536.16, 3,655.78, 4,281.14, 4,930.28, 5,843.94, and 5,942.00 and at least one antibody to one or more proteins, protein fragments, or peptides of the group consisting of m/z values of 902.48, 1,479.76, 2,407.30, 2,536.16, 3,655.78, 4,281.14, 4,930.28, 5,843.94, and 5,942.00

20. A method according to claim 1 wherein a doubly charged biomarker characteristic of the Sjögren's Syndrome fingerprint is selected from a group in the m/z range of 3,803 to 3,808 and at least one antibody to one or both of the said biomarkers.
